(19) United States
(12) Patent Application Publication
(10) Pub. No.: US 2012/0065534 A1
(43) Pub. Date: Mar. 15, 2012

(54) DIAGNOSTIC NANOSENSOR DEVICE AND METHOD FOR BREATH ANALYSIS

(75) Inventor: Anastasia RIGAS, Setauket, NY (US)

(73) Assignee: AMDT Inc., Setauket, NY (US)

(21) Appl. No.: 13/226,082

(22) Filed: Sep. 6, 2011

Related U.S. Application Data
(60) Provisional application No. 61/379,963, filed on Sep. 3, 2010.
(60) Provisional application No. 61/452,391, filed on Mar. 14, 2011.
(60) Provisional application No. 61/452,507, filed on Mar. 14, 2011.

(51) Int. Cl. A61B 5/097

(52) U.S. Cl. 600/532

(57) ABSTRACT

A hand-held portable device detects the concentration of gas such as ammonia, and displays the concentration, by whether it exceeds a threshold and/or in numerical concentration.

### CROSS-REFERENCE TO RELATED APPLICATIONS

The present application claims priority on provisional application U.S. Ser. No. 61/379,963, filed Sep. 3, 2010, which is incorporated by reference herein. This application also incorporates by reference herein U.S. Ser. No. 61/452,391 filed Mar. 14, 2011, and U.S. Ser. No. 61/452,507 filed Mar. 14, 2011.

### BACKGROUND OF THE INVENTION

The present invention relates to a diagnostic breath test device based using a nanosensor and using signal analysis of the nanosensor response to a gas, such as ammonia NH₃, in a breath sample.

Some of the many ways to detect certain diseases in a patient are biopsy, histological exam, antibody detection, antigen detection and breath test. A breath test device has been used to detect Helicobacter pylori or H. pylori. Otsuka America Pharmaceutical has marketed a device called BreathTek™, a ¹³C-urea breath test (labeled urea), which relies on the detection of ¹³CO₂ in breath using IR spectroscopy. For the BreathTek™ urea breath test (commercially available test), the patient provides a baseline breath sample, ingests 75 mg ¹³C-urea, and 15 min later provides a second breath sample. Both samples are sent to a central lab, which determines the concentration of ¹³CO₂ in each of them and reports the results one week later. Test results are reported as positive or negative and a value is also provided, described as Delta over Baseline i.e., the difference between the ratio ¹³CO₂/¹²CO₂ in the post-urea sample and the baseline sample. A test is positive when the second breath sample is enriched in ¹³CO₂ beyond a threshold value (“Delta Over Baseline”≧2.4). The company reports the test results as either positive or negative and also provides the numerical value of the Delta over Baseline.

Some of the limitations or disadvantages of such a device are that the test results are not immediate, and that the samples are sent to a laboratory. Also, this test device uses labeled urea which adds cost.

### SUMMARY OF THE INVENTION

The present invention provides a breath test device and method which can be implemented in a hand-held self-contained device and provide immediate results, so that the diagnosis can be obtained quickly, and so that treatment of a detected disease can commence immediately.

The invention provides a hand-held portable device comprising: a detector for detecting the concentration of gas in a breath sample, and a display device for providing an output indicating the concentration.

The invention provides a method of detecting the concentration of gas in a breath sample, comprising: detecting the concentration of gas in a breath sample using a hand-held portable device, and displaying an output indicating the concentration with a display device.

### BRIEF DESCRIPTION OF THE DRAWING FIGURES

FIG. 1 shows a device for breath analysis;

FIG. 2 shows another device for breath analysis;

FIG. 3 shows a device for NH₃ assessment in breath air;

FIG. 4 is a graph showing how current changes at NH₃ 50-80 ppb;

FIG. 5 (left and right) are graphs showing the response of a sensor using an optimized set-up;

FIG. 6 shows two urea breath tests (UBT) analyzed using a method according to the invention;

FIG. 7 shows UBT results using a method and device according to the invention (ammonia score) and for the BreathTek™ device; and

FIG. 8 shows electronic circuitry of a device according to the invention.

### DETAILED DESCRIPTION OF THE PREFERRED EMBODIMENT

A description of a preferred embodiment and method will be described, but the invention is not limited to this embodiment.

The invention provides a hand-held portable device comprising: a detector for detecting the concentration of gas in a breath sample, and a display device for providing an output indicating the concentration.

The detector may detect the concentration of ammonia. The detector may be a nanosensor. The display device may indicate whether the concentration of gas detected is above or below a threshold concentration. The display device may indicate the concentration of gas detected in a quantitative numerical amount. The device may further include a memory for storing the concentrations of gas detected for at least two breath samples. The device may further include a computation unit for computing the difference in concentration of gas in the two gas samples. The display device may provide an output indicating the difference in concentration in the two gas samples. The sensor may have a characteristic resistance value which changes depending on the concentration of gas product. The detector may further comprise a Wheatstone bridge having one leg in the form of a sensor whose resistance varies depending on the gas concentration. The device may further comprise a trap to trap at least one other gas from reaching the detector. The trap may be a CO₂/H₂O trap. The detector may comprise a sensor which detects the concentration of gas and provides a sensor signal having a characteristic which indicates the amount of gas detected, an acquisition module which converts the characteristic into a digital value, and a memory/computation unit which compares the digital value to it previously stored threshold value. The acquisition module may comprise an A/D converter. The detector may produce two component signals, including a positive signal and a negative signal, wherein the amplitude of the negative signal indicates the concentration of gas.

The invention provides a method of detecting the concentration of gas in a breath sample, comprising: detecting the concentration of gas in a breath sample using a hand-held portable device, and displaying an output indicating the concentration with a display device.

The step of detecting may comprise detecting ammonia. The step of detecting may comprise using a nanosensor. The step of displaying may comprise displaying whether the concentration of gas detected is above or below a threshold amount. The step of displaying may comprise displaying the concentration of gas detected as a quantitative amount. The method may further include detecting a first breath sample as a baseline; administering urea to the patient; and detecting a second breath sample after the urea administration. The method may further comprise computing the difference in concentrations detected between the two breath samples. The method may further comprise displaying the difference in concentrations detected. The method may further comprise using a trap to trap at least one other gas component before the step of detecting. The step of detecting may comprise using a sensor which detects the concentration of gas and provides a sensor signal having a characteristic which indicates the amount of gas detected, and comparing the amount detected to a threshold value.

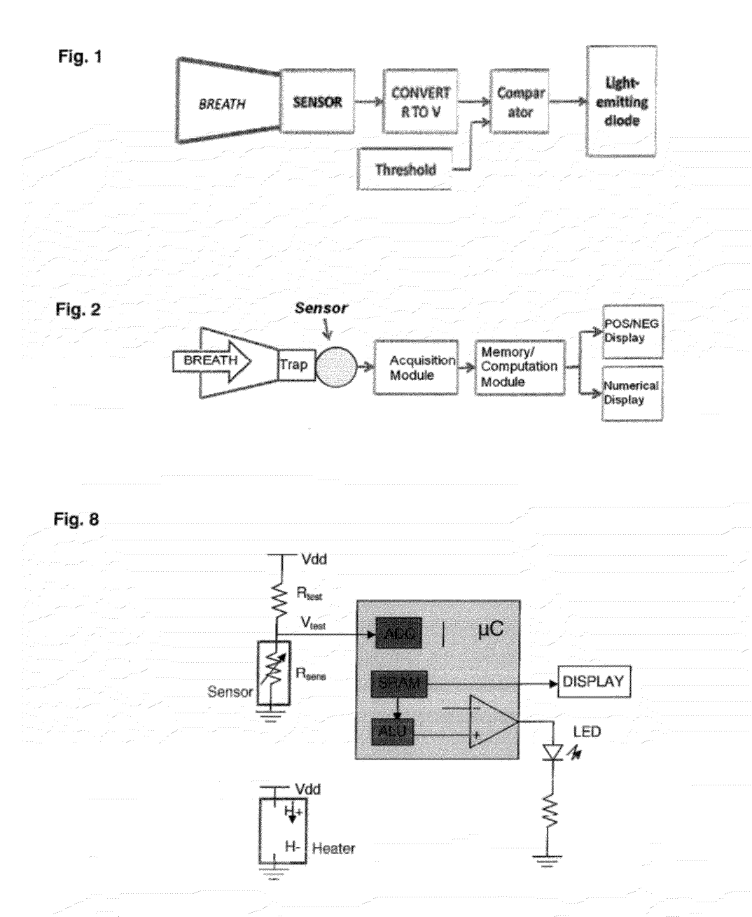

FIG. 1 shows a device for breath analysis, comprising a sensor which is shown receiving a breath sample and which has a resistor which changes its resistance R value depending on the detected amount of NH₃, a converter which converts the R value to a V (voltage) signal, a comparator which compares the voltage output of the converter to a threshold value, and a light emitting diode LED. When the breath sample encounters the sensor, the concentration of ammonia NH₃ is determined. If the concentration exceeds a predetermined threshold, the LED is turned on, indicating that the concentration NH₃ exceeds a threshold amount.

As shown in FIG. 2, a device according to the invention comprises a mouthpiece for receiving a breath sample, a trap which traps CO₂/H₂O, a sensor which detects NH₃, an acquisition module which converts the sensor signal into a digital value, a memory/computation module which stores the digital sensor readout in a memory unit and determines the region in which the system is operating, and contains the threshold value NH₃ breath levels for the binary response, and two displays, including a POS/NEG display, and a numerical display which displays the detected concentration of NH₃ in units. The sensor includes a Wheatstone bridge, with one leg having a sensor whose resistance varies as a function of the amount of NH₃ detected.

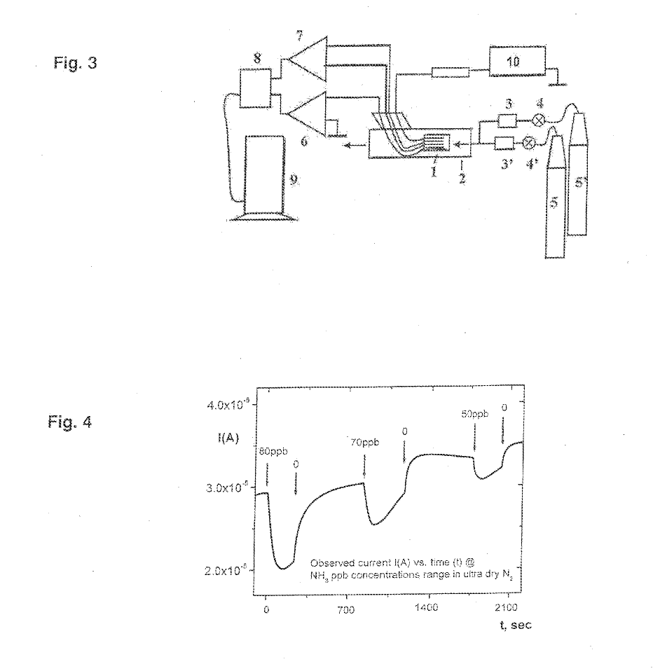

In the development of the device, a sensor was tested, and the gas-sensing properties of the sensor were evaluated, as shown in FIG. 3. Initially, a gas flow bench was designed and manufactured to assess the sensitivity, linearity, bias stability, thermal stability and regeneration rate of the gas sensor. Electrical signals processed by the DC technique were digitized with acquisition boards. In a typical assay, the test gas, e.g., NH₃ in dry N₂, released from the gas cylinder, reaches the sensor 1 inside the test chamber 3. If the gas reacts significantly with the sensor, i.e., the gas is absorbed by the sensor, the resistance of the sensor changes, which changes the current generated by the power supply 10. This change in current is converted into a voltage signal by the current preamplifier 6. The output signal is then digitized by the analog-digital (A/D) converter 8 and displayed and stored in the computer 9. Other components are: digital flow controllers 3, 3′; valves and pressure regulators 4, 4′; gas cylinders 5, 5′; and voltage preamplifier 7.

To evaluate the sensitivity of the sensor, it was exposed to low concentrations of NH₃ gas in dry as described above. NH₃ was detected easily down to 50 ppb as shown in FIG. 4. Of note, NH₃ concentrations encountered in human breath during UBT testing are 100-200 ppb, i.e., within the detection range.

FIG. 4 shows a plot of Current I (A) changes at NH₃ 50-80 ppb. Pulses of NH₃ in ultra-dry N₂ were tested using this setup. The current was recorded at constant voltage applied to the sensor. The detected current variation was plotted against time. The changes in current response reflect the changes in NH₃ concentrations.

The sensor specificity was evaluated by exposing it to NO₂, NO, C₃H₆, and H₂, each one of which may be encountered in human breath and interfere with NH₃ determination. At 440° C., the sensor was very sensitive to NH₃, generating a response approximately 20 times greater than from the other gases at concentrations up to 100 ppm.

Because human breath contains H₂O and CO₂ (up to 5%), their influence on NH₃ sensing was determined. Under the test conditions, both interfered with NH₃ sensing. To overcome this limitation, a Decarbite (PW Perkins and Co) CO₂ filter was used, which reacts only with highly acidic gases such as CO₂ and H₂S, thus excluding the possibility of cross adsorption. Using Decarbite as a desiccant trap, neither CO₂ nor H₂O affected NH₃ sensing. Because of these results, the desiccant was incorporated into the device.

Since the trap also partially suppressed NH₃ sensing, a) the interaction time was increased of the gas being analyzed with the sensor, and b) its injection rate was increased up to 100 seem (standard cubic centimeters per minute) while decreasing the flow rate of N₂ to ˜300 scent. (These conditions approximate the flow characteristics of forcefully exhaled human breath.) Under these conditions, low concentrations of NH₃ still provided a low signal. The signal conditioning circuit was modified from simple 4-point (or 2-point) resistance measurements to the Wheatstone bridge geometry. This modification eliminated the baseline and amplified the signal greatly, making it both easily detectable and analyzable.

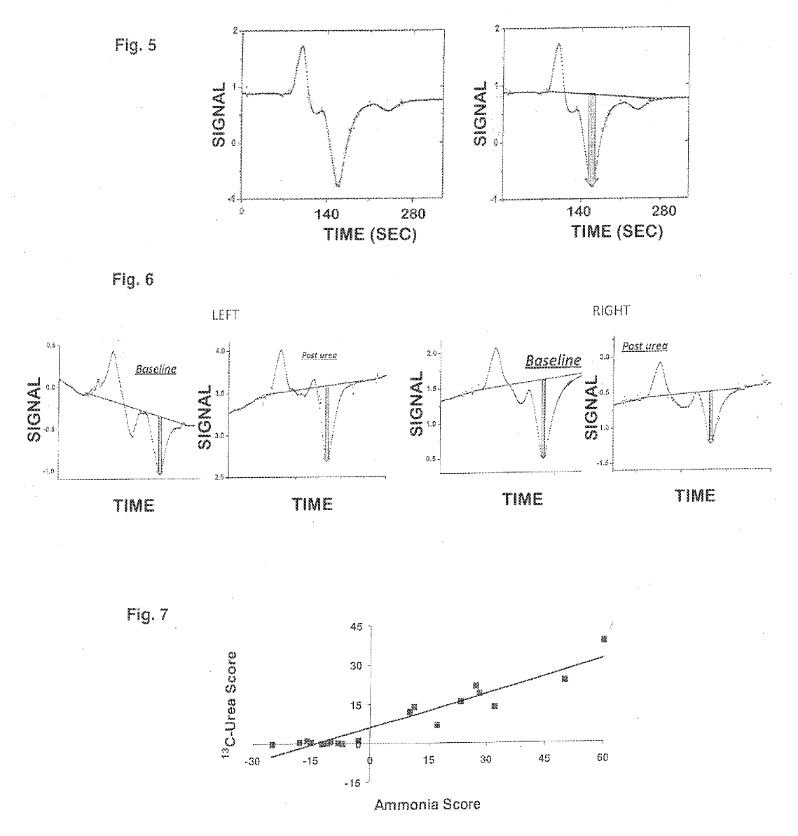

The Wheatstone bridge generates a signal with two components; one positive and one negative (FIG. 5). The positive component is due to the cooling down of the sensor in response to the flow of the gas pulse in the presence of the gas carrier flow. The negative component of the signal is due to NH₃ (see also FIG. 6). When the sample gas reaches the sensor, the resistance changes, generating the negative component of the signal. Thus, in our assays of NH₃, the amplitude of only the negative component was determined, the only one responding to changes in NH₃ concentration.

FIG. 5 shows the response of the sensor using the optimized set-up. FIG. 5 Left shows the signal generated after the sensor was exposed to NH₃ in N₂ 100 ppm. The positive and negative components of the signal are clearly identified. The signal (ordinate) represents voltage, but for practical purposes the amplitude of the peak was treated as dimensionless. FIG. 5 Right shows, in the same tracing, the baseline is extrapolated and an arrow indicates the amplitude of the negative peak (used to calculate the response of the sensor to NH₃).

The device was subjected to human testing. The approach was used to evaluate 20 patients recruited at Stony Brook University Hospital (IRB #123855-1). Patients were studied undergoing BreathTek™ UBT for their medical evaluation and not for this study. This test has been extensively validated, by being compared to UBTs based on: mass spectrometry, a different IR method, the Meretek UBT®, and endoscopy-based methods for H. Pylori diagnosis. The overall agreement of BreathTek™ with each of these methods was 99.06%-99.55%. Thus, the results from the BreathTek™ device were used to assess the results of the breath test device herein.

For the study, immediately after each sample was obtained for the commercial test, the patient provided two more breath samples, each immediately after those for the commercial BreathTek™ test. Using the present device and method, the concentration of NH₃ was determined. In analyzing the results, we determined the amplitude of the negative component of the signal was determined before and after urea ingestion and the difference in amplitude (A) between the two was calculated, generating the “Ammonia Score”: Ammonia Score=A_{after}−A_{before} A positive Ammonia Score value indicates a positive breath test, and a negative value indicates a negative breath test. FIG. 6 shows examples of a positive and a negative result obtained with the present device and method. Similar to the commercial test, each breath test was characterized as either positive or negative and the value of the Ammonia Score was determined.

FIG. 5 shows two UBTs analyzed with the present device and method (breath NH₃ assay). FIG. 5 Left shows a positive test wherein the post urea arrow is bigger than baseline. FIG. 5 Right shows a negative test wherein the post urea arrow is smaller than baseline.

For the 20 patients evaluated, the results by the present device and method were compared to those obtained with the commercial breath test. In all cases, there was perfect (100%) agreement between the present device and the commercial BreathTek™ as to which test was positive or negative. Furthermore, there was a statistically highly significant correlation between the numerical values obtained from each patient by the two methods (R=0.947; p<0.001) (FIG. 7).

FIG. 7 shows UBT results using the present device and method (Ammonia Score) and BreathTek™ (¹³C-Urea Score). The correlation between them was excellent. The BreathTek™ has a narrow range of values for the negative tests, and could be considered to be less accurate.

The present device for ammonia sensing is based on the principle that the response of the sensor will modify a characteristic of an electronic circuit, e.g., electrical resistance, or its inverse which is conductance. The resistance of the sensor is a function of NH₃ concentration. The resistance of the sensor is first converted to a voltage signal. The voltage signal generated by the sensor is then compared to predefined threshold voltage values. These values, set through a variable resistor, determine the region in which the system is operating. The diode display provides a visual indication in the form of a red (above threshold) or green light, depending on the outcome. The reproducibility of the measurements has been confirmed over 1 week by daily measurements of a standard gas mixture.

The conductance of the sensor is proportional to NH₃ concentration. To sense the concentration of NH₃, the resistance of the sensor is first converted to a voltage signal. After the first (baseline) breath test, the voltage signal is converted through an analog-to-digital converter to a digital value, which will be displayed. The digital value would also be stored in memory.

When the second breath sample is analyzed, the voltage signal is again converted to a digital value and displayed. The change in the concentration of NH₃ is computed, based on the two values, using a micro-controller. This change is displayed as the final numerical result. For a binary response, the change in NH₃ content will be compared to a predefined threshold value. The threshold voltage value would be set through calibration measurements and stored as a digital value in the microcontroller.

FIG. 8 shows the electronic circuitry of the device. FIG. 8 shows a sensor, interface circuitry and display. A micro-controller (μC) contains the Analog-to-Digital Converter, memory (SRAM), and an Arithmetic Logic Unit (ALU). The Vₜₛₑₜ is a voltage proportional to the resistance of the sensor. The sensor may be a metal-oxide nanosensor for detecting NH₃. If one wishes to detect gases other than NH₃, other sensors could be used. More than one sensor could be incorporated, with a switch to select connection of the sensor to the circuit for the specific gas to be detected.

The device and method of the invention can be used for diagnostic purposes in humans (and animals) with infectious diseases, cancers, metabolic diseases, liver diseases, kidney diseases, endocrine diseases, nervous system diseases, and bone diseases, by way of example and not limitation. The invention could be used to analyze gases in breath sample to diagnose and prevent above named diseases.

The invention could also be used to detect other and to all gases contained in human breath, animal breath, room air, and car exhaust, for example.

Although one preferred embodiment has been described, the invention is not limited to this embodiment, and scope of the invention is defined by the appended claims.

1. A hand-held portable device comprising:
a detector for detecting the concentration of gas in a breath sample, and
a display device for providing an output indicating the concentration.

2. The device according to claim 1, wherein the detector detects the concentration of ammonia.

3. The device according to claim 1, wherein the detector is a nanosensor.

4. The device according to claim 1, wherein the display device indicates whether the concentration of gas detected is above or below a threshold concentration.

5. The device according to claim 1, wherein the display device indicates the concentration of gas detected in a quantitative numerical amount.

6. The device according to claim 1, further including a memory for storing the concentrations of gas detected for at least two breath samples.

7. The device according to claim 6, further including a computation unit for computing the difference in concentration of gas in the two gas samples.

8. The device according to claim 7, wherein the display device provides an output indicating the difference in concentration in the two gas samples.

9. The device according to claim 1, wherein the sensor has a characteristic resistance value which changes depending on the concentration of gas product.

10. The device according to claim 1, wherein the detector further comprises a Wheatstone bridge having one leg in the form of a sensor whose resistance varies depending on the gas concentration.

11. The device according to claim 1, wherein the device further comprises a trap to trap at least one other gas from reaching the detector.

12. The device according to claim 11, wherein the trap is a CO₂/H₂O trap.

13. The device according to claim 1, wherein the detector comprises a sensor which detects the concentration of gas and provides a sensor signal having a characteristic which indicates the amount of gas detected, an acquisition module which converts the characteristic into a digital value, a memory/computation unit which compares the digital value to a previously stored threshold value.

14. The device according to claim 13, wherein the acquisition module comprises an A/D converter.

15. The device according to claim 1, wherein the detector produces two component signals, including a positive signal and a negative signal, wherein the amplitude of the negative signal indicates the concentration of gas.

16. A method of detecting the concentration of gas in a breath sample, comprising:
detecting the concentration of gas in a breath sample using a band-held portable device, and
displaying an output indicating the concentration with a display device.

17. The method according to claim 16, wherein the step of detecting comprises detecting ammonia.

18. The method according to claim 16, wherein the step of detecting comprises using a nanosensor.

19. The method according to claim 16, wherein the step of displaying comprises displaying whether the concentration of gas detected is above or below a threshold amount.

20. The method according to claim 16, wherein the step of displaying comprises displaying the concentration of gas detected as a quantitative amount.

21. The method according to claim 16, further including:
detecting a first breath sample as a baseline;
administering urea to the patient; and
detecting a second breath sample after the urea administration.

22. The method according to claim 20, further comprising computing the difference in concentrations detected between the two breath samples.

23. The method according to claim 21, further comprising displaying the difference in concentrations detected.

24. The method according to claim 16, further comprising using a trap to trap at least one other gas component before the step of detecting.

25. The method according to claim 16, wherein the step of detecting comprises using a sensor which detects the concentration of gas and provides a sensor signal having a characteristic which indicates the amount of gas detected, and comparing the amount detected to a threshold value.
